(19) United States
(12) Patent Application Publication
(10) Pub. No.: US 2010/0081149 A1
(43) Pub. Date: Apr. 1, 2010

(54) NOVEL OXIDIZED LDL COMPLEX AND METHOD FOR DETECTION THEREOF

(75) Inventor: Eiji Matsuura, Okayama (JP)

Correspondence Address: SWANSON & BRATSCHUN, L.L.C., 8210 SOUTHPARK TERRACE, LITTLETON, CO, 80120, US

(73) Assignee: Okayama Prefecture Industrial Promotion Foundation, Okayama (JP)

(21) Appl. No.: 12/302,638

(22) PCT Filed: May 29, 2007
(86) PCT No.: PCT/JP2007/060887

§ 371 Date: Nov. 26, 2008

(30) Foreign Application Priority Data
May 30, 2006 (JP) 2006-149430

(51) Int. Cl. G01N 33/543; C07K 14/435

(52) U.S. Cl. 435/7.21; 530/395

(57) ABSTRACT

Serum amyloid P component, oxidized LDL and β2-glycoprotein I can together form a complex. The presence of a disease such as hyperlipemia and atherosclerosis can be determined by detecting the complex by using either one of an anti-serum amyloid P component antibody and an anti-β2-glycoprotein I antibody.

### TECHNICAL FIELD

The present invention relates to a complex comprising serum amyloid P component, oxidized LDL, and β2-glycoprotein I, a method for detecting the same, and a kit used for the same.

### BACKGROUND ART

Abbreviations used in the present application and descriptions thereof are as follows.

### β₂-GPI: β2-Glycoprotein I

BSA: Bovine serum albumin
CRP: C-reactive protein
EDTA: Ethylenediamine tetraacetic acid
ELISA: Enzyme-linked immunosorbent assay
HRP: Horseradish peroxidase
LDL: Low density lipoprotein (unoxidized native LDL not)

### OD: Absorbance

oxLDL: Oxidized LDL
oxLDL/β₂-GPI complex: A complex comprising oxLDL and β₂-GPI
PBS: Phosphate-buffered physiological saline
SAP: Serum amyloid P component (serum amyloid P component, one of pentraxin proteins)
SAP/oxLDL/β₂-GPI complex: A complex comprising SAP, oxLDL, and β₂-GPI
SAP/oxLDL complex: A complex comprising SAP and oxLDL
TBS: Tris-buffered physiological saline

SAP is a one of acute phase proteins induced upon inflammation in mice (Non-Patent Document 1). Meanwhile, oxLDL forms a complex with β₂-GPI, and this complex of oxLDL and β₂-GPI is known to further form a complex with CRP in humans (Patent Document 1).

However, it has not been known that SAP forms a complex with oxLDL and β₂-GPI. Let alone this, it has not been known either that this complex can be used as a marker of a disease. Furthermore, it has not been known that SAP forms a complex with oxLDL, or that this complex can be used as a marker of a disease.

### [Patent Document 1] JP-A-2004-271502

[Non-Patent Document 2] Carlanda C, Bottazzi B, Bastone A, and Mantovani A. Pentraxins at the crossroads between innate Immunity, inflammation, matrix deposition, and female fertility. Annu Rev Immunol 23: 337-66, 2005

### DISCLOSURE OF THE INVENTION

### Problems to be Solved by the Invention

An object of the present invention is to provide a SAP/oxLDL/β₂-GPI complex and a SAP/oxLDL complex, a method for detecting the same, a kit used for the same, and the like.

### Means for Solving the Problems

The present inventors researched relationships among SAP, oxLDL, and β₂-GPI. As a result, they surprisingly found that these three molecules formed a complex and provided this complex, thus accomplishing the present invention. Furthermore, the inventors provided a method and a kit for detecting this complex and a method and kit for detecting a disease through detection of this complex, thus accomplishing the present invention.

Specifically, the present invention provides a SAP/oxLDL/β₂-GPI complex (hereinafter referred to as “the complex of the present invention”).

Furthermore, the present invention provides a method for detecting the complex of the present invention, comprising a step of bringing at least one of an anti-SAP antibody and an anti-β₂-GPI antibody into contact with a sample that may contain the complex of the present invention (hereinafter referred to as “the method for detecting the complex of the present invention”). Examples of the method for detecting the complex of the present invention include a method for detecting the complex of the present invention, comprising a step of bringing an anti-SAP antibody and an anti-β₂-GPI antibody into contact with a sample that may contain the complex of the present invention to form a sandwich complex comprising “anti-SAP antibody—the complex of the present invention—anti-β₂-GPI antibody”. Preferably, one of the anti-SAP antibody and the anti-β₂-GPI antibody in the method for detecting the complex of the present invention is immobilized on a solid phase.

Furthermore, examples of the method for detecting the complex of the present invention include a method for detecting the complex of the present invention, comprising at least the following steps (1) to (3):

step (1), a step of bringing a sample that may contain the complex of the present invention into contact with a solid phase on which antibody A is immobilized to form a first complex represented by “antibody A immobilized on a solid phase—the complex of the present invention”;

step (2), a step of bringing antibody B into contact with the first complex formed in step 1 to form a sandwich complex represented by “antibody A immobilized on a solid phase—the complex of the present invention—-antibody B”; and

step (3), a step of detecting the sandwich complex formed in step 2 (wherein, the antibody A represents one of an anti-SAP antibody and an anti-β₂-GPI antibody, and the antibody B represents the other antibody).

The “sample that may contain the complex of the present invention” mentioned above is preferably a sample derived from an organism. Furthermore, this “sample derived from an organism” is preferably a body fluid.

Furthermore, the present invention provides a method for detecting a disease, comprising at least the following steps (1) and (2) (hereinafter referred to as “the disease detection method of the present invention”):

step (1), a step of detecting the complex of the present invention present in a body fluid by the method for detecting the complex of the present invention; and

step (2), a step of associating a detection result obtained in step (1) and a disease.

The “disease” mentioned here is preferably selected from hyperlipemia and atherosclerosis.

Furthermore, the present invention provides a kit for detecting the complex of the present invention, comprising at least an anti-SAP antibody and an anti-β₂-GPI antibody as components thereof (hereinafter referred to as “the kit for detecting the complex of the present invention”). Preferably, one of the anti-SAP antibody and the anti-β₂-GPI antibody is immobilized on a solid phase.

Furthermore, the present invention provides a kit for detecting a disease, comprising the kit for detecting the complex of the present invention (hereinafter referred to as “the disease detecting kit of the present invention”). This “disease” is preferably selected from hyperlipemia and atherosclerosis.

At the same time, the inventors found that SAP and oxLDL formed a complex and provided this complex, thus accomplishing the present invention. Furthermore, the inventors provided a method and a kit for detecting this complex and a method and a kit for detecting a disease through detection of this complex, thus accomplishing the present invention.

Specifically, the present invention further provides a SAP/oxLDL complex (hereinafter referred to as “the complex of the present invention 2”).

Furthermore, the present invention provides a method for detecting the complex of the present invention 2, comprising a step of bringing at least either an anti-SAP antibody or an anti-oxLDL antibody into contact with a sample that may contain the complex of the present invention 2 (hereinafter referred to as “the method for detecting the complex of the present invention 2”). Examples of the method for detecting the complex of the present invention 2 include a method for detecting the complex of the present invention 2, comprising a step of bringing an anti-SAP antibody and anti-oxLDL antibody into contact with a sample that may contain the complex of the present invention 2 to form a sandwich complex comprising “anti-SAP antibody—the complex of the present invention 2—anti-oxLDL antibody”. Preferably, one of the anti-SAP antibody and the anti-oxLDL antibody in the method for detecting the complex of the present invention 2 is immobilized on a solid phase.

Furthermore, examples of the method for detecting the complex of the present invention 2 include a method for detecting the complex of the present invention 2, comprising at least the following steps (1) to (3):

step (1), a step of bringing a sample that may contain the complex of the present invention 2 into contact with a solid phase on which antibody C is immobilized to form a first complex represented by “antibody C immobilized on a solid phase—the complex of the present invention 2”;
step (2), a step of bringing antibody D into contact with the first complex formed in step 1 to form a sandwich complex represented by “antibody C immobilized on a solid phase—the complex of the present invention 2—antibody D”; and

step (3), a step of detecting the sandwich complex formed in step 2 (wherein, the antibody C represents one of an anti-SAP antibody and an anti-oxLDL antibody, and the antibody D represents the other antibody).

The “sample that may contain the complex of the present invention 2” mentioned above is preferably a sample derived from an organism. Furthermore, this “sample derived from an organism” is preferably a body fluid.

Furthermore, the present invention provides a method for detecting a disease, comprising at least the following steps (1) and (2) (hereinafter referred to as “the disease detection method of the present invention 2”):

step (1), a step of detecting the complex of the present invention 2 present in a body fluid by the method for detecting the complex of the present invention 2; and

step (2), a step of associating a detection result in step (1) and a disease.

The “disease” mentioned here is preferably selected from hyperlipemia and atherosclerosis.

Furthermore, the present invention provides a kit for detecting the complex of the present invention 2 including an anti-SAP antibody and an anti-oxLDL antibody as components thereof (hereinafter referred to as “the kit for detecting the complex of the present invention 2”). Preferably, one of the anti-SAP antibody and the anti-oxLDL antibody is immobilized on a solid phase.

Furthermore, the present invention provides a kit for detecting a disease, comprising the kit for detecting the complex of the present invention 2 (hereinafter referred to as “the disease detecting kit of the present invention 2”). This “disease” is preferably selected from hyperlipemia and atherosclerosis.

### Advantages of the Invention

The complex of the present invention is very useful because it can be used as, for example, a standard for the method for detecting the complex of the present invention, the disease detection method of the present invention, the kit for detecting the complex of the present invention, and the disease detecting kit of the present invention. Furthermore, the method for detecting the complex of the present invention is very useful because, according to this method, the complex of the present invention present in a sample can be detected conveniently, rapidly, and easily with high sensitivity and high precision at a low cost, and the kit for detecting the complex of the present invention is provided based on this method. The disease detection method of the present invention is very useful because a disease such as hyperlipemia or atherosclerosis can be detected through detection of the SAP/oxLDL/β₂-GPI complex present in a body fluid conveniently, rapidly, and easily with high sensitivity and high precision at a low cost, and the disease detecting kit of the present invention is further provided based on this method. Furthermore, the kit for detecting the complex of the present invention and the disease detecting kit of the present invention are very useful because the method for detecting the complex of the present invention and the disease detection method of the present invention can be used more conveniently, rapidly, and easily by using these kits. Furthermore, these present inventions can also be used in the situation of drug development such as exploring a candidate substance of a drug for a disease (for example, hyperlipemia and atherosclerosis) using a model animal of the disease and evaluating efficacy of the substance.

Furthermore, the complex of the present invention 2, the method for detecting the complex of the present invention 2, the disease detection method of the present invention 2, the kit for detecting the complex of the present invention 2, and the disease detecting kit of the present invention 2 are also very useful as with the complex of the present invention, the method for detecting the complex of the present invention, the disease detection method of the present invention, the kit for detecting the complex of the present invention, and the disease detecting kit of the present invention described above.

### BEST MODE FOR CARRYING OUT THE INVENTION

Hereafter, embodiments of the present invention will be explained.

### <1> Complex of the Present Invention

The complex of the present invention is a SAP/oxLDL/β₂-GPI complex. The complex of the present invention is characterized by comprising SAP, oxLDL, and β₂-GPI.

The complex of the present invention can be produced by bringing three molecules, SAP, oxLDL, and β₂-GPI, into contact under physiological conditions. SAP, oxLDL, and β₂-GPI can be produced by respective known methods. Furthermore, those commercially available can also be used.

The “physiological conditions” are not particularly limited so long as the conditions are comparable with the salt concentration, pH, temperature, and the like in the body of a mouse (in particular, blood). Such conditions are, for example, physiological saline having a buffer action, pH 6 to 8 (preferably pH 6.5 to 7.5), and temperature of 35 to 40° C. (preferably 36 to 38° C.).

Furthermore, the method of “contact” in the present application is not particularly limited so long as molecules to be brought into contact become in contact with each other.

Furthermore, the order of bringing SAP, oxLDL, and β₂-GPI into contact is not particularly limited. For example, these three molecules may be brought into contact at the same time, or any two thereof may be brought into contact, and then the remaining one may be brought into contact. For example, a method can be mentioned in which oxLDL and β₂-GPI are brought into contact, and then SAP is brought into contact therewith.

The quantity ratio of these molecules at the time of contact is not particularly limited either, and can be suitably selected by those skilled in the art. For example, when a method is adopted in which oxLDL and β₂-GPI are brought into contact, and then SAP is brought into contact therewith, the mass ratio of oxLDL and β₂-GPI is oxLDL: β₂-GPI=1:5 to 5:1 (preferably 1:4 to 4:1, more preferably 1:3 to 3:1, more preferably 1:2 to 2:1). Then, when SAP is brought into contact, the mass ratio of the oxLDL/β₂-GPI complex and SAP is, for example, oxLDL/β₂-GPI complex:SAP=20:1 to 1:10 (preferably 15:1 to 1:5, more preferably 12:1 to 1:2). In this case, SAP is preferably brought into contact in the presence of calcium ions having a final concentration of approx. 0.1 to 10 mM (preferably approx. 0.5 to 5 mM).

Furthermore, the durations of these contacts are not particularly limited either, and can be suitably selected by those skilled in the art. Since a reaction is time-dependent, and sufficient complexes are formed with a longer contact time, approx. 10 to 48 hours (preferably 12 to 24 hours) can be mentioned, for example. See the examples described later for more specific methods for producing the complex of the present invention.

Furthermore, the complex of the present invention can also be produced by purifying it from blood of a hyperlipemia model mouse given a high fat diet (high fat diet-loaded). Specifically, the complex of the present invention can be produced by feeding a high fat diet to a hyperlipemia model mouse (for example, LDLR−/− mouse or apoE−/− mouse, described later), then collecting blood, and purifying the complex of the present invention from this blood.

The “purification” mentioned here is a concept encompassing not only so-called complete purification, but also partial purification. The purification method is not particularly limited either. For example, a technique such as affinity chromatography using an anti-SAP antibody or an anti-β₂-GPI antibody can be used. Known anti-SAP antibody or anti-β₂-GPI antibody can be used in this technique. It is needless to say that those commercially available may be used. For example, as shown in the examples described later, anti-SAP antibodies are commercially available and can be used. Furthermore, as an anti-β₂-GPI antibody, “WB-CAL-1” (J. Immunol., 149, p1063-1068 [1992]) can be used, for example.

The complex of the present invention can be used as a standard for, for example, the method for detecting the complex of the present invention, the disease detection method of the present invention, the kit for detecting the complex of the present invention, and the disease detecting kit of the present invention. For example, quality of detection of the complex of the present invention by these detecting methods and detecting kits can be checked using the complex of the present invention. Furthermore, by creating a calibration curve or a relational expression for the relationship between the complex of the present invention having a known concentration and a detection result of ELISA, the complex of the present invention having an unknown concentration contained in a sample can be quantified. It is needless to say that the complex of the present invention can be used as a component of each kit of the present invention.

### <2> Method for Detecting the Complex of the Present Invention

The method for detecting the complex of the present invention is a method for detecting the complex of the present invention, comprising a step of bringing at least either an anti-SAP antibody or an anti-β₂-GPI antibody into contact with a sample that may contain the complex of the present invention.

Here, “a sample that may contain the complex of the present invention” means a sample in which the complex of the present invention is intended to be detected. So long as there is such an intention, whether the complex of the present invention is actually contained in the sample or not does not matter.

Furthermore, the type of the “sample” is not particularly limited either. The sample may be artificially produced or derived from a natural substance. For example, an aqueous solution of an artificially produced complex of the present invention and a sample derived from an organism fall within the scope of the “sample” mentioned here. Above all, samples derived from an organism are preferred, and fluids derived from an organism (body fluids) are preferred. The type of the body fluid is not particularly limited either. Examples thereof include blood. The “blood” mentioned here is a concept encompassing serum, plasma, and the like.

The method for detecting the complex of the present invention is characterized by comprising a step of bringing at least either an anti-SAP antibody or an anti-β₂-GPI antibody into contact with such a sample.

Therefore, an anti-SAP antibody alone may be brought into contact with such a sample, an anti-β₂-GPI antibody alone may be brought into contact, or both of these antibodies may be brought into contact. Furthermore, when both of the antibodies are brought into contact, both of them may be brought into contact at the same time, or one of them is first brought into contact and then the other one is brought into contact, as described above. When the method of bringing both of them into contact is adopted, only the complex of the present invention can be specifically detected even if foreign substances such as SAP, β₂-GPI, and the oxLDL/β₂-GPI complex exist in a sample. The anti-SAP antibody and the anti-β₂-GPI antibody used here are not particularly limited so long as they are antibodies binding to SAP and β₂-GPI, respectively. However, antibodies specifically binding to these antigens are preferred to increase the precision of detection. These antibodies may be either monoclonal antibodies or polyclonal antibodies. However, taking into account mass production and uniformity, monoclonal antibodies are preferred.

These antibodies can be prepared according to usual antibody preparation methods using these antigens. Furthermore, commercially available antibodies may be used as they are. For example, commercially available anti-SAP antibodies can be used as they are. Furthermore, specific examples of the anti-β₂-GPI antibody include WB-CAL-1 (J. Immunol., 149, p1063-1068 [1992]), Cof-22 and Cof-23 (Blood, 87, p3262-3270 [1996]), and EY2C9 (Arthritis Rheum., 37, p1453-1461 [1994]).

Furthermore, the condition of the “contact” is not particularly limited so long as it is a condition under which these antibodies and the complex of the present invention yield antigen-antibody reactions, and can be suitably selected by those skilled in the art.

The method for detecting the complex of the present invention may further comprise other steps so long as it comprises such steps. For example, it may further comprise a step of preparing a sample, a step of washing, a step of performing a reaction for detecting the complex of the present invention, a step of creating a calibration curve or a relational expression, and a step of analyzing a detection result of the complex of the present invention.

In the present application, “detection” is a concept encompassing both qualitative detection (detection of presence or absence or existence or nonexistence) and quantitative detection (detection of amount of presence or degree). Therefore, for example, measurement of the amount (concentration) of the complex of the present invention falls within the scope of the “detection” mentioned here.

Furthermore, the specific method for detecting the complex of the present invention is not particularly limited either so long as it comprises the above-mentioned steps. Examples of the specific method include immunological measurement techniques using antibodies (ELISA [sandwich method, competition method, inhibition method, etc.], immunoblotting, agglutination method, etc.).

Examples of the method for detecting the complex of the present invention include a method for detecting the complex of the present invention, comprising a step of bringing an anti-SAP antibody and an anti-β₂-GPI antibody into contact with a sample that may contain the complex of the present invention to form a sandwich complex comprising “anti-SAP antibody—the complex of the present invention—anti-β₂-GPI antibody”. Preferably, one of the anti-SAP antibody and the anti-β₂-GPI antibody used in the method for detecting the complex of the present invention is immobilized on a solid phase.

Furthermore, examples of the method for detecting the complex of the present invention also include a method for detecting the complex of the present invention, comprising at least the following steps (1) to (3):

step (1), a step of bringing a sample that may contain the complex of the present invention into contact with a solid phase on which antibody A is immobilized to form a first complex represented by “antibody A immobilized on a solid phase—the complex of the present invention”;

step (2), a step of bringing antibody B into contact with the first complex formed in step 1 to form a sandwich complex represented by “antibody A immobilized on a solid phase—the complex of the present invention—antibody B”; and

step (3), a step of detecting the sandwich complex formed in step 2 (wherein the antibody A represents one of an anti-SAP antibody and an anti-β₂-GPI antibody, and the antibody B represents the other antibody).

Hereafter, each step will be explained.

Step (1) is a step of bringing a sample that may contain the complex of the present invention into contact with a solid phase on which antibody A is immobilized to form a first complex represented by “antibody A immobilized on a solid phase—the complex of the present invention”.

The “solid phase on which antibody A is immobilized” mentioned here can be produced by immobilizing antibody A on a solid phase.

The solid phase used to immobilize this antibody A is not particularly limited so long as it can immobilize antibody A and is insoluble in water, body fluids, measurement reaction mixtures, and the like. Examples of the form of the solid phase include plates (for example, wells of a microplate), tubes, beads, membranes, and gels. Examples of material of the solid phase include polystyrene, polypropylene, nylon, and polyacrylamide. Of these, plates made of polystyrene as material are preferred.

As a method for immobilizing antibody A on these solid phases, common methods for immobilizing proteins or lipids such as physical adsorption methods and covalent bonding methods can be used. Among these methods, physical adsorption methods are preferred because their procedures are simple, and these methods are often used. Specific examples of physical adsorption methods include a method comprising dissolving antibody A in a buffer or the like, bringing this solution into contact with a solid phase (for example, microplate), and allowing antibody A to be adsorbed to a solid phase.

Furthermore, antibody A may not be immobilized on some parts of the surface of a solid phase on which antibody A is immobilized. The complex of the present invention, a foreign substance molecule, or the like in the sample that can be immobilized thereon may affect a result of detection. Therefore, it is preferable to add a blocking substance to coat the part on which antibody A is not immobilized before bringing a sample into contact with a solid phase. As such blocking substances, serum albumin, casein, skim milk, gelatin, or other commercially available blocking substances can be used.

By bringing a sample that may contain the complex of the present invention into contact with the “solid phase on which antibody A is immobilized” obtained as described above, the first complex represented by “antibody A immobilized on a solid phase—the complex of the present invention” is formed. The “contact” mentioned here is not particularly limited so long as the molecule of the complex of the present invention in the sample and the molecule of antibody A immobilized on the solid phase are in contact with each other. The condition of the “contact” is not particularly limited so long as antibody A and the complex of the present invention yield an antigen-antibody reaction under this condition as described above, and can be suitably selected by those skilled in the art.

Step (2) is a step of bringing antibody B into contact with the first complex formed in step 1 to form a sandwich complex represented by “antibody A immobilized on a solid phase—the complex of the present invention—antibody B”.

By bringing antibody B into contact with the first complex formed in step 1, a sandwich complex represented by “antibody A immobilized on a solid phase—the complex of the present invention—antibody B” is formed.

The “contact” mentioned here is not particularly limited either so long as the molecule of antibody B and the first complex molecule (immobilized on a solid phase) are in contact with each other. The condition of the “contact” is not particularly limited either so long as antibody B and the first complex molecule (immobilized on a solid phase) yield an antigen-antibody reaction as described above, and can be suitably selected by those skilled in the art.

The antibody A refers to one of an anti-SAP antibody and an anti-β₂-GPI antibody, and the antibody B refers to the other antibody. Therefore, when an anti-SAP antibody is used as antibody A, antibody B is an anti-β₂-GPI antibody. On the contrary, when an anti-β₂-GPI antibody is used as antibody A, antibody B is an anti-SAP antibody.

Step (3) is a step of detecting the sandwich complex formed in step 2.

This method for “detecting the sandwich complex formed in step 2” is not particularly limited either so long as it can detect the sandwich complex comprising “antibody A immobilized on a solid phase—the complex of the present invention—antibody B,” but detection is preferably performed by detecting “antibody B” existing at an open end (the end not directly immobilized on the solid phase) of the sandwich complex.

To this end, the “antibody B” brought into contact in step 2 itself may be labeled with a labeling substance beforehand. Furthermore, when a substance that is not labeled as “antibody B” is used, a substance binding to “antibody B” and labeled with a labeling substance may be used. Examples of the “substance binding to ‘antibody B’” include an antibody that specifically binds to the immunoglobulin depending on the animal from which “antibody B” (immunoglobulin) is derived, class thereof, or the like. For example, when “antibody B″ is a sheep-derived IgG, an anti-sheep IgG antibody can be used as a “substance binding to ‘antibody B’”.

“Antibody B” in the sandwich complex formed in step 2 comprising “antibody A immobilized on a solid phase—the complex of the present invention—antibody B” can be detected by detecting this labeling substance. That is, this sandwich complex is detected.

Examples of such labeling substances used for labeling include enzymes (peroxidase, alkaline phosphatase, β-galactosidase, luciferase, acetylcholine esterase, etc.), fluorescent dyes (fluorescein isothiocyanate (FITC), etc.), chemiluminescence substances (luminol, etc.), biotin, and avidin (including streptavidin), and they are not particularly limited so long as they can be used for usual protein labeling. The labeling method can be suitably selected from known methods suitable for labeling substances such as, for example, glutaraldehyde methods, periodate crosslink methods, maleimide crosslink methods, carbodiimide methods, and activated ester methods (refer to “Chemistry of proteins [II],” TOKYO KAGAKU DOJIN CO., LTD., 1987). For example, the method can be suitably selected from methods using a hydrazide derivative of biotin (refer to Avidin-Biotin Chemistry: A Handbook, 57-63, PIERCE CHEMICAL COMPANY, 1994) when biotin is used as a labeling substance, and the method can be suitably selected from the methods described in JP-B-63-17843 and the like when fluorescein isothiocyanate is used.

To detect a labeling substance, those skilled in the art can suitably select a detection method depending on the labeling substance used. For example, when peroxidase is used as a labeling substance, detection can be performed by adding a chromogenic substrate such as tetramethylbenzidine or o-phenylene diamine as a substrate of the enzyme and aqueous hydrogen peroxide and determining the degree of coloration of the product by an enzymatic reaction examining a change in absorbance. Furthermore, when a fluorescent substance or a chemiluminescent substance is used, methods of measuring fluorescence or luminescence of a solution after a reaction can be used.

### <3> Disease Detection Method of the Present Invention

The disease detection method of the present invention is a disease detection method, comprising at least the following steps (1) and (2):

step (1), a step of detecting the complex of the present invention present in a body fluid by the method for detecting the complex of the present invention; and

step (2), a step of associating a detection result in step (1) and a disease.

Hereafter, each step will be explained.

Step (1) is a step of detecting the complex of the present invention present in a body fluid by the method for detecting the complex of the present invention.

The disease detection method of the present invention is an application of the method for detecting the complex of the present invention to a disease detection method. For “the method for detecting the complex of the present invention” in step (1), see the above “<2> Method for detecting the complex of the present invention”. Here, however, a “body fluid” is used as “a sample that may contain the complex of the present invention”. The “body fluid” used here is not particularly limited so long as it is derived from an animal to be detected for a disease. The animal to be detected for a disease is not particularly limited, and examples thereof include animals other than human, such as mouse, rat, and rabbit.

Furthermore, the type of the “body fluid” is not particularly limited so long as the amount of the complex of the present invention contained in the body fluid is changed by the disease, and blood (a concept encompassing serum and plasma) is preferred.

Step (2) is a step of associating a detection result in step (1) and a disease. By this step, a disease can be detected.

As described above, the term “detection” in the present application is a concept encompassing not only qualitative detection but also quantitative detection. Therefore, the “detection result in step (1)” mentioned here may be “presence or absence or existence or nonexistence” of the complex of the present invention in a body fluid (qualitative detection result) or “amount of presence or degree” (quantitative detection result). Furthermore, the “amount of presence” or “degree” may be an amount obtained from a calibration curve drawn using a standard with known concentrations, a relational expression, or the like (actual measurement value) or a ratio to that in healthy animals (animals not having the disease) (relative value).

Furthermore, the amount of the complex of the present invention in a body fluid can be increased by a disease. In this case, when the amount of the complex in a body fluid is larger than that in healthy animals, the result can be associated with a finding of “having a disease” or “being likely to have a disease”. When the amount of the complex in a body fluid is equal to that in healthy animals, the result can be associated with a finding “not having a disease” or “not being likely to have a disease”.

Furthermore, the disease detection method of the present invention includes not only detection of presence or absence of a disease but also detection of the severity of the disease. For example, when the amount of the complex in a body fluid of an animal is measured, and the amount of the complex tends to increase, the result can be associated with a finding “the disease is progressing” or “the disease is very likely to be progressing”. On the contrary, when the measured amount of the complex tends to decrease, the result can be associated with a finding “the disease is improving” or “the disease is very likely to be improving”. Furthermore, when the measured amount of the complex does not change, the result can be associated with a finding “there is no change in the severity of the disease (or degree of healthiness)” or “it is very likely that there is no change in the severity of the disease (or degree of healthiness)”. Therefore, the disease detection method of the present invention can be used for exploration of candidate substances of a drug, efficacy evaluation of a substance, and the like.

The “disease” detected by the disease detection method of the present invention is not particularly limited so long as the amount of the complex of the present invention in a body fluid is changed by the disease, but is preferably selected from hyperlipemia and atherosclerosis.

As described above, a disease can be detected by detecting the complex of the present invention present in a body fluid by the method for detecting the complex of the present invention and associating a detection result with a disease.

### <4> Kit for Detecting the Complex of the Present Invention

The kit for detecting the complex of the present invention is a kit for detecting for the complex of the present invention, comprising at least an anti-SAP antibody and an anti-β₂-GPI antibody as components.

The anti-SAP antibody and the anti-β₂-GPI antibody that can be used here are the same as those described in the above “<2> Method for detecting the complex of the present invention”. Preferably, one of the anti-SAP antibody and the anti-β₂-GPI antibody is immobilized on a solid phase. The solid phase and the method for immobilizing an antibody that can be used here are the same as described in the above “<2> Method for detecting the complex of the present invention”.

The kit for detecting the complex of the present invention may further comprise other components so long as it comprises an anti-SAP antibody and an anti-β₂-GPI antibody as components.

Examples of other components that can be added to the kit for detecting the complex of the present invention include a reagent for detecting a labeling substance and “an antibody that binds to an anti-SAP antibody or an anti-β₂-GPI antibody and is labeled with a labeling substance (secondary antibody)”. In addition to these components, a blocking substance, a wash, a solution for diluting a sample, a solution for terminating an enzymatic reaction, and the like may be comprised.

These components are placed in separate containers and can be stored as a kit that can be used by the method for detecting the complex of the present invention when used. Detection of the complex of the present invention using the kit for detecting the complex of the present invention can be performed according to the method for detecting the complex of the present invention. Above all, the kit is preferably used in the method comprising detecting the complex by forming a sandwich complex (a so-called sandwich method: see the above “<2> Method for detecting the complex of the present invention”).

### <5> Disease Detecting Kit of the Present Invention

The disease detecting kit of the present invention is a disease detecting kit comprising the kit for detecting the complex of the present invention. See the above section for the kit for detecting the complex of the present invention.

The “disease” mentioned here is preferably selected from hyperlipemia and atherosclerosis. Detection of a disease using the disease detecting kit of the present invention can be performed according to the disease detection method of the present invention.

### <6> Complex of the Present Invention 2

The complex of the present invention 2 is a SAP/oxLDL complex. The complex of the present invention 2 is characterized by comprising two molecules, SAP and oxLDL.

The complex of the present invention 2 can be produced by bringing SAP and oxLDL into contact with each other under physiological conditions. SAP and oxLDL can both be produced by respective known methods. Furthermore, those commercially available can also be used.

The “physiological conditions” mentioned here are the same as described in the above <1>.

Furthermore, the ratio of these two molecules when brought into contact with each other is not particularly limited and can be suitably selected by those skilled in the art. Examples of the mass ratio of oxLDL and SAP include oxLDL:SAP=20:1 to 1:10 (preferably 15:1 to 1:5, more preferably 12:1 to 1:2). In this case, SAP is preferably brought into contact in the presence of calcium ion having a final concentration of about 0.1 to 10 mM (preferably about 0.5 to 5 mM).

The contact time of these molecules and other points are the same as described in the above <1>.

The complex of the present invention 2 can be used as, for example, a standard for the method for detecting the complex of the present invention 2, the disease detection method of the present invention 2, the kit for detecting the complex of the present invention 2, and the disease detecting kit of the present invention 2. For example, quality of detection of the complex of the present invention 2 by these detection methods or detecting kits can be checked using the complex of the present invention 2. Furthermore, for example, when a calibration curve or a relational expression is created for the relationship between the complex of the present invention 2 having known concentrations and a detection result by ELISA, the complex of the present invention 2 having an unknown concentration contained in a sample can be quantified. It is needless to say that the complex of the present invention 2 can be used as a component of each kit of the present invention.

### <7> Method for Detecting the Complex of the Present Invention 2

The method for detecting the complex of the present invention 2 is a method for detecting the complex of the present invention 2, comprising a step of bringing at least either an anti-SAP antibody or an anti-oxLDL antibody into contact with a sample that may contain the complex of the present invention 2.

The “sample that may contain the complex of the present invention 2,” the “sample,” and the like are the same as explained in the above <2>.

The method for detecting the complex of the present invention 2 is characterized by comprising a step of bringing at least either an anti-SAP antibody or an anti-oxLDL antibody into contact with such a sample.

The anti-oxLDL antibody is not particularly limited so long as it is an antibody binding to oxLDL. A monoclonal antibody or a polyclonal antibody can be prepared using oxLDL as an antigen according to a usual antibody preparation method. Of these, a mouse monoclonal anti-oxLDL antibody is preferably used.

Others are the same as explained in the above <2>.

Examples of the method for detecting the complex of the present invention 2 include a method for detecting the complex of the present invention 2, comprising a step of bringing an anti-SAP antibody and an anti-oxLDL antibody into contact with a sample that may contain the complex of the present invention 2 to form a sandwich complex comprising “anti-SAP antibody—the complex of the present invention 2-anti-oxLDL antibody”. Preferably, one of the anti-SAP antibody and the anti-oxLDL antibody in the method for detecting the complex of the present invention 2 is immobilized on a solid phase.

Furthermore, examples of the method for detecting the complex of the present invention 2 also include a method for detecting the complex of the present invention 2, comprising at least the following steps (1) to (3):

step (1), a step of bringing a sample that may contain the complex of the present invention 2 into contact with a solid phase on which antibody C is immobilized to form a first complex represented by “antibody C immobilized on a solid phase—the complex of the present invention 2”;

step (2), a step of bringing antibody D into contact with the first complex formed in step 1 to form a sandwich complex represented by “antibody C immobilized on a solid phase—the complex of the present invention 2—antibody D”; and

step (3), a step of detecting the sandwich complex formed in step 2,

(wherein, the antibody C refers to one of anti-SAP antibody and an anti-oxLDL antibody, and the antibody D refers to the other antibody).

These steps are the same as explained in the above <2>. These steps can be understood by replacing the “antibody A” and the “antibody B” in the above <2> with “antibody C” and “antibody D”. Furthermore, the “anti-β₂-GPI antibody” in the above <2> can be replaced with “anti-oxLDL antibody”.

### <8> Disease Detection Method of the Present Invention 2

The disease detection method of the present invention 2 is a disease detection method, comprising at least the following steps (1) and (2):

step (1), a step of detecting the complex of the present invention 2 present in a body fluid by the method for detecting the complex of the present invention 2; and

step (2), a step of associating a detection result in step (1) and a disease.

The disease detection method of the present invention 2 is the same as explained in the above <3>.

<9> Kit for detecting the complex of the present invention 2

The kit for detecting the complex of the present invention 2 is a kit for detecting the complex of the present invention 2, comprising at least an anti-SAP antibody and an anti-oxLDL antibody as components.

The kit for detecting the complex of the present invention 2 is the same as explained in the above <4>. The kit can be understood by replacing “anti-β₂-GPI antibody” in the above <4> with “anti-oxLDL antibody”.

### <10> Disease Detecting Kit of the Present Invention 2

The disease detecting kit of the present invention 2 is a disease detecting kit, comprising the kit for detecting the complex of the present invention 2. See above for the kit for detecting the complex of the present invention 2.

The “disease” mentioned here is preferably selected from hyperlipemia and atherosclerosis. Detection of a disease using the disease detecting kit of the present invention 2 can be performed according to the disease detection method of the present invention 2.

Hereafter, the present invention will be specifically explained with reference to the examples.

### Example 1

### Preparation of SAP/oxLDL/β₂-GPI Complex and Establishment of Assay System

### Materials and Methods

(1) Hyperlipemia Model Mice (LDLR−/−, apoE−/−)

In this example, LDLR−/− mice and apoE−/− mice (C57BL/6 mice background; purchased from The Jackson Laboratory, U.S.) were used as atherosclerosis-predisposed hyperlipemia model mice. These are mice deficient in a receptor (LDLR) and a ligand (apoE), respectively, associated with LDL metabolism. Since virtually no LDL is present in blood of a mouse, the mouse is naturally an animal free from atherosclerosis. In these hyperlipemia model mice, however, a chylomicron-remnant, a precursor of LDL, and IDL (intermediate [low]-density lipoprotein) are not taken up into the liver or tissues, and LDL can be allowed to remain in blood. Therefore, hyperlipemia and atherosclerosis can be easily developed by giving a high fat diet to these LDLR−/− mice and apoE−/− mice to elevate blood cholesterol levels (Proc. Natl. Acad. Sci. U.S.A., 98, p7946-51 [2001]; Proc. Natl. Acad. Sci. U.S.A., 91, p4431-5 [1994]).

(2) Preparation of Mouse and Human oxLDL

To plasma of an LDLR−/− or apoE−/− hyperlipemia model mouse which had been given a high fat diet (Oriental Yeast Co., Ltd.) for 2 weeks or fresh human plasma was added 200 mM EDTA at a final concentration of 0.25 mM to obtain EDTA-containing plasma. 750 μL of the EDTA-containing plasma was added into a 3-mL ultracentrifuge tube (3.5 PC, Beckman, Fullerton, Calif.), 250 μL of PBS (containing 0.25 mM EDTA) was overlaid, and the mixture was centrifuged at 100,000 rpm and 10° C. for 7 min (TL-100 rotor; TLA100.3, Beckman). Then, 250 μL of the supernatant was removed, 250 μL of PBS (containing 0.25 mM EDTA) was overlaid, and the mixture was further centrifuged under the same condition for 2.5 h. Then, 250 μL of the supernatant was removed, and 150 μL of PBS containing KBr was mixed. Then, the mixture was centrifuged under the same condition for 5 h, and 200 μL of the supernatant was recovered to obtain an LDL fraction. The obtained LDL fraction was dialyzed overnight against PBS (containing 1 mM EDTA) and then oxidized.

LDL was oxidized by incubating LDL having a final concentration of 100 μg/mL at 37° C. in the presence of 5 μM copper sulfate. At 12 h after the start of incubation, EDTA was added at a final concentration of 1 mM to terminate the oxidation reaction. The oxidized fraction was dialyzed against PBS (containing 1 mM EDTA) and then concentrated, and the oxidation degree was checked with a thiobarbituric acid-reactive substance (TBARS) (J. Lipid Res., 28, p495-509 [1987]).

(3) Antibody (anti-β₂-GPI Antibody)

As an anti-β₂-GPI antibody, “WB-CAL-1” (IgG2a, κ) was used. This antibody is a β₂-GPI-reactive monoclonal autoantibody. It is an antibody derived from an anti-phospholipid antibody syndrome model mouse (NZWxBXSB mouse), does not react with free β₂-GPI, and shows reactivity to β₂-GPI forming a complex with oxLDL. This antibody recognizes human and mouse β₂-GPI in a complex (Immunol., 149, p1063-1068 [1992]).

### (4) ELISA (Assay of Mouse and Human SAP/oxLDL/β₂-GPI Complexes)

First, 8 μg/mL of WB-CAL-1 was added to an Immulon 2HB plate (Thermo Labsystems) at 50 μL/well and incubated overnight at 4° C. to solid-phase WB-CAL-1 on the plate. After solid-phasing, 200 μL/well of TBS containing 0.05% Tween20 (containing 1.25 mM Ca²⁺) was added, and the plate was washed 3 times.

Subsequently, the plate was blocked with TBS containing 0.5% BSA (containing 1.25 mM Ca²⁺). The plate was washed in the same manner as described above, and then a sample diluted to an appropriate concentration was added to each well and incubated overnight at 27° C. Then, the plate was washed in the same manner as described above.

Subsequently, a solution of sheep anti-mouse SAP antibody (or rabbit anti-human SAP antibody) 10,000-fold diluted was added at 100 μL/well and incubated at room temperature for 1 h. Then, the plate was washed in the same manner as described above.

Subsequently, a solution of HRP-labeled anti-sheep IgG antibody (or anti-rabbit IgG antibody) 4000-fold diluted was added at 100 μL/well and incubated at room temperature for 1 h. Then, the plate was washed in the same manner as described above.

Subsequently, 100 μL of the o-PD reagent solution (4 mg of o-phenylenediamine dihydrochloride/10 mL of 0.1 M citrate buffer (pH 5.0) and 10 μL of 30% H₂O₂) was added to each well and incubated at room temperature for 20 min. Then, 1 M H₂SO₄ was added at 100 μL/well to terminate the reaction, and absorbance was measured at 490 nm.

### (5) Preparation of Mouse and Human SAP/oxLDL/β₂-GPI Complexes and Drawing of Standard Curve of the Complexes

First, a reference standard of the SAP/oxLDL/β₂-GPI complex was prepared as follows. First, mouse or human-derived oxLDL and β₂-GPI were mixed in PBS at a mass ratio of 1:1, and the mixture was incubated overnight at 37° C. Then, calcium ion was added to the complex solution at a final concentration of 1 mM, mouse or human-derived SAP was mixed therein at a mass ratio of oxLDL/β₂-GPI complex and SAP=10:1, and the mixture was incubated overnight at 37° C. The final concentration of the complex was adjusted to 0.1 mg/mL as oxLDL.

The mouse or human SAP/oxLDL/β₂-GPI complex thus obtained was diluted serially, and absorbance at 490 nm was measured by ELISA described above. As controls, the oxLDL/β₂-GPI complex and SAP were similarly diluted serially, and absorbance was measured simultaneously with the above-mentioned complex. A standard curve was drawn for the relationship between the concentration of the mouse or human SAP/oxLDL/β₂-GPI complex and the measurement results.

### (6) Changes in Mouse SAP/oxLDL/β₂-GPI Complex Formation Over Time

SAP was mixed with the mouse oxLDL/β₂-GPI complex (mixed at a mass ratio of 4:1 and incubated overnight at 37° C.) in the presence of calcium ion (mass ratio of oxLDL/β₂-GPI complex and SAP=20:1), and a reaction was started at 37° C. A predetermined amount of the sample was collected at 1, 2, 4, and 8 h after the start of the reaction and then frozen at −80° C. to terminate the reaction. The remaining sample was continuously reacted, and the reaction was terminated at 24 h after the start of reaction.

To investigate changes in the formation of the mouse SAP/oxLDL/β₂-GPI complex over time, this sample was examined by ELISA and electrophoresis. The method of ELISA is as described above.

Furthermore, electrophoresis was performed by spotting of 6 to 7 μg of the sample on an agarose gel film (Helena Laboratories Corp.) and applying 90 V in 0.05 M barbital buffer (pH 8.6) for 40 min. Two types of staining, lipid staining and protein staining, were performed. Fat Red 7B (Sigma-Aldrich) was used for the lipid staining, and Amido Black 10B (Nacalai Tesque, Inc.) was used for protein staining.

### (7) Detection of Complex in Serum of High Fat Diet Mouse

A high fat diet was given to 27 LDLR−/− hyperlipemia model mice for 2 weeks, and then whole blood was collected. The collected blood was recovered in a heparin-added tube and centrifuged at 10,000 rpm and 4° C. for 10 min to separate plasma, which was used as a sample to quantify the SAP/oxLDL/β₂-GPI complex by the above-mentioned ELISA. As controls, serum samples of BALB/c mice and C57BL/6 mice were used.

### (8) Reliability Assurance Tests of Assay System Dilution Linearity Test

Of the plasma samples collected from the hyperlipemia model mice (Apoe−/− mice and LDLR−/− mice) given a high fat diet, samples positive for the SAP/oxLDL/β₂-GPI complex were diluted serially, and the SAP/oxLDL/β₂-GPI complex was measured by the above-mentioned ELISA. Correlations between the dilution factors and the measured values were examined to check that the complex had been quantified concentration-dependently and accurately.

### Recovery Test

The reference standard (having a known concentration) of the SAP/oxLDL/β₂-GPI complex was diluted with serum (derived from BALB/c mice) negative for the SAP/oxLDL/β₂-GPI complex serially. The SAP/oxLDL/β₂-GPI complex in this sample was measured by the above-mentioned ELISA, and the recovery was calculated to check that the expected amount was detected.

### Within-Run Reproducibility Test

Using the plasma samples positive for the SAP/oxLDL/β₂-GPI complex in the hyperlipemia model mice given a high fat diet, the SAP/oxLDL/β₂-GPI complex was measured by subjecting 10 wells of the same sample to the above-mentioned ELISA. Distribution from the mean to each value was examined by calculating coefficient of variation (CV) of the measured values.

### Results

### (1) Standard Curve of Mouse SAP/oxLDL/β₂-GPI Complex

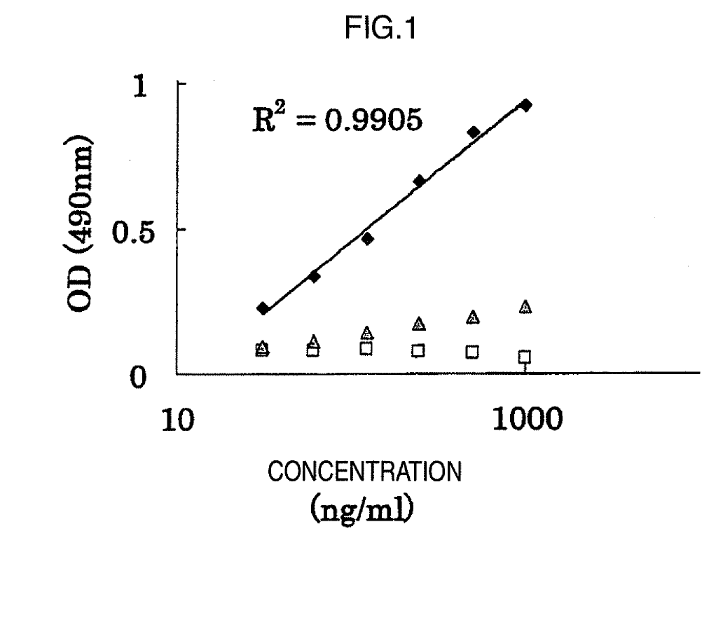

The mouse SAP/oxLDL/β₂-GPI complex, the mouse oxLDL/β₂-GPI complex, and the mouse SAP alone were serially diluted and subjected to measurement. The results are shown in FIG. 1. In FIG. 1, black squares represent the measured values (logarithmic values) of the SAP/oxLDL/β₂-GPI complex, white squares represent the measured values of the oxLDL/β₂-GPI complex, and triangles represent the measured values of SAP alone. The concentration of the SAP/oxLDL/β₂-GPI complex was expressed by a concentration as LDL protein.

As shown in FIG. 1, favorable linearity was confirmed between the concentrations and the measured values of the SAP/oxLDL/β₂-GPI complex in a double logarithmic graph, and a standard curve could be drawn.

Furthermore, cross-reactivity between the oxLDL/β₂-GPI complex and SAP alone was not observed in this assay system. Therefore, it was demonstrated that the SAP/oxLDL/β₂-GPI complex could be specifically detected in this assay system.

### (2) Changes in Formation of SAP/oxLDL/β₂-GPI Complex Over Time

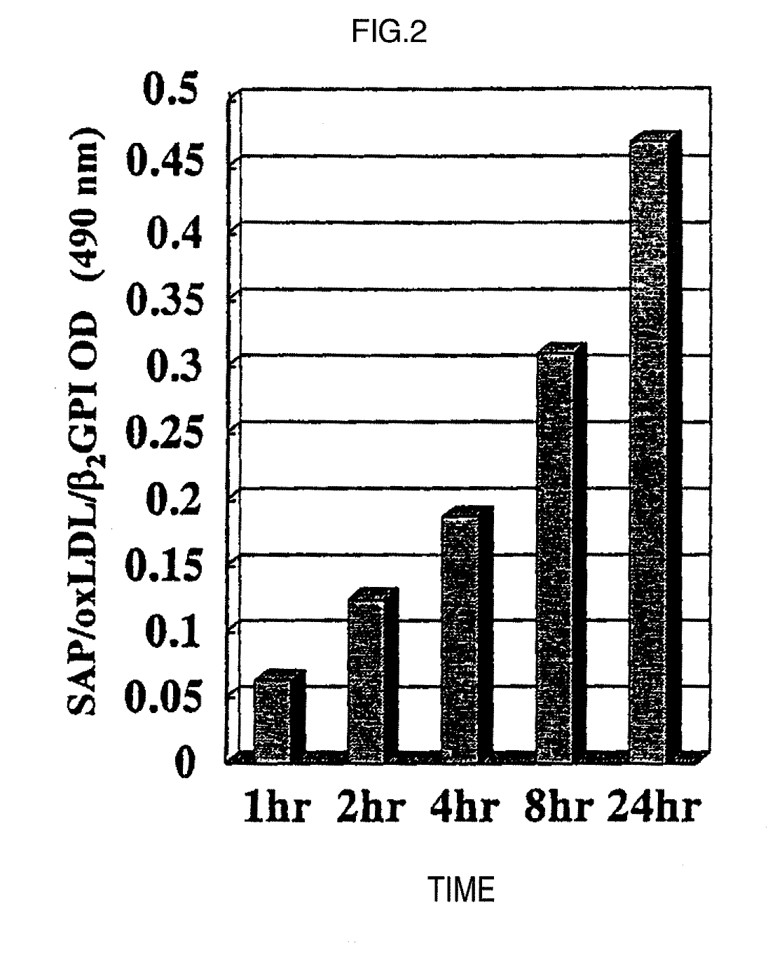

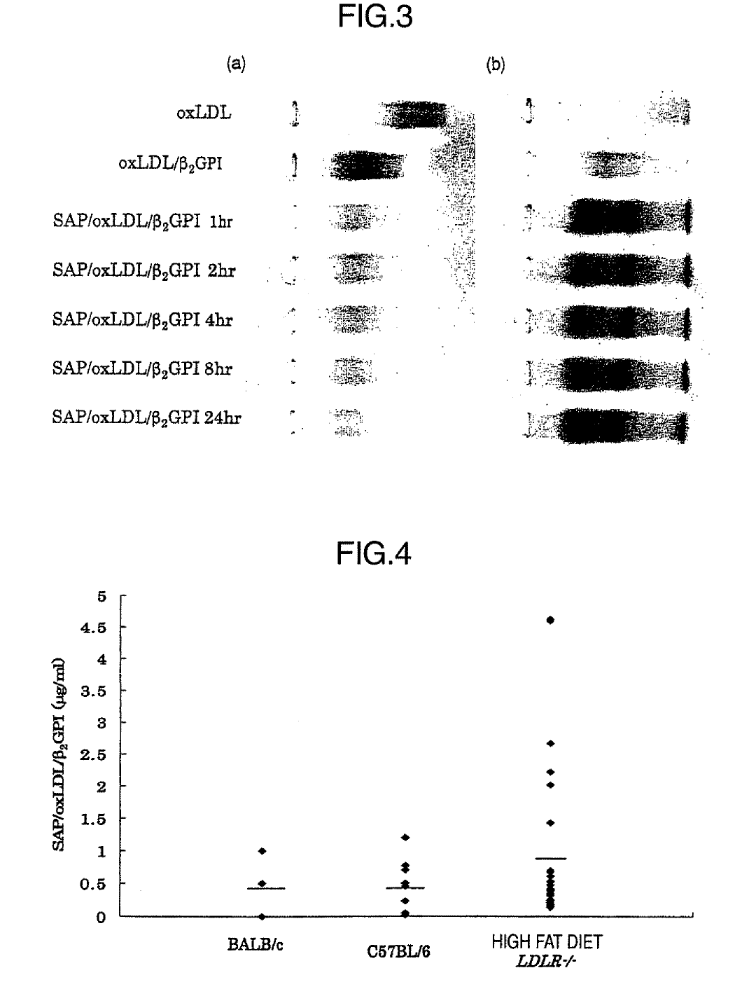

To observe changes in formation of the SAP/oxLDL/β₂-GPI complex over time, the SAP/oxLDL/β₂-GPI complex in samples prepared by changing the reaction time for complex formation was checked by ELISA and electrophoresis. The results of ELISA are shown in FIG. 2, and the results of electrophoresis are shown in FIG. 3. (a) in FIG. 3 shows the results of Fat Red 7B staining, and (b) shows the results of Amide Black staining. In FIG. 3, oxLDL and oxLDL/β₂-GPI were also subjected to electrophoresis at the same time for comparison.

FIG. 2 shows that the concentration of the SAP/oxLDL/β₂-GPI complex increases (complex formation was progressing) with the reaction time.

As shown in FIG. 3, it is known that, when β₂-GPI binds to oxLDL, a negative charge is eliminated, and mobility of the complex is decreased as compared with oxLDL alone in electrophoresis. Changes in mobility by binding SAP to this oxLDL/β₂-GPI complex were observed to examine changes in complex formation over time. As a result, both Fat Red 7B staining, which stains lipids, and Amide Black staining, which stains proteins, showed that the migration distance towards the anode (the right side of FIG. 3 is anode) was gradually decreased with the reaction time. Therefore, it was shown that the negative charge of oxLDL was eliminated with the complex formation. Furthermore, two thick bands were confirmed near the center in Amide Black staining. This appears to be because complement component C3 originally existed in SAP (reagent) and was detected.

### (3) Detection of SAP/oxLDL/β₂-GPI Complex in Mice Given High Fat Diet (High Fat Fed) (16 to 24 Weeks Old)

Using plasma collected from LDLR−/− mice that had been given a high fat diet (high fat fed LDLR−/− mice) for 2 weeks and serum samples collected from BALB/c mice and C57BL/6 mice at the same age (both controls) as samples, the SAP/oxLDL/β₂-GPI complex was measured by ELISA. Increased atherosclerosis lesions (plaques) started being observed in the hyperlipemia model mice at the same age. The results are shown in FIG. 4.

FIG. 4 showed that some samples from the hyperlipemia model mice given a high fat diet (high fat fed LDLR−/− mice) had high values. Since the mean concentration of the SAP/oxLDL/β₂-GPI complex in this sample is higher than in the control groups, it was suggested the possibility of existence of the SAP/oxLDL/β₂-GPI complex in the hyperlipemia model mice given a high fat diet.

### (4) Reliability Tests of Assay System

To check reliability of the ELISA assay system, the following tests were performed. Favorable results were obtained in all the tests, and reliability of this assay system was confirmed.

### Dilution Linearity Test

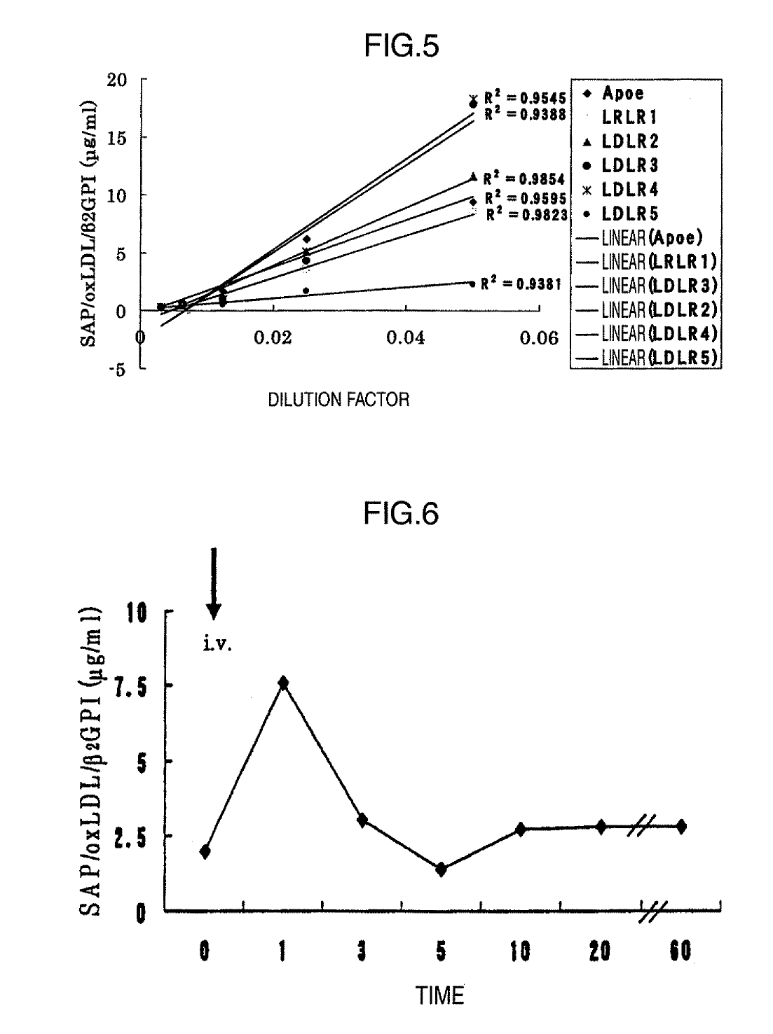

The results are shown in FIG. 5. Correlations between the dilution factors and the measured values were examined. As a result, all the samples showed the correlation coefficient (R² value) close to 1, showing a favorable linear relationship.

### Recovery Test

The results are shown in Table 1. The recovery rates were obtained by the following two calculation methods as “Recovery 1” and “Recovery 2”. The following “A” to “E” refer to parts shown as “A” to “E,” respectively, in Table 1.

(D)=((A−B)/C)×100 Recovery 1

(E)=(A/(B+C))×100 Recovery 2

As a result, since the recovery rates were almost 100%, it was demonstrated that the expected amounts were measured by this ELISA assay system.

### Within-Run Reproducibility Test

The results are shown in Table 2. CV (coefficient of variation) shows distribution of each measured value from the mean. In general, when the CV value is within 10%, a favorable result is determined. As shown in Table 2, the CV values of all the samples were within 10%, and favorable results were obtained.

TABLE 1

| Amount added | Serum + | | Recovery1 | Recovery2 |
|---|---|---|---|---|
| (ng/ml) | complex | minus blank | (%) | (%) |
| 0 | B 658 | 0 | | |
| C 62.5 | A 701 | 43 | D 68.8 | E 97.3 |
| 125 | 791 | 133 | 106.4 | 101 |
| 250 | 936 | 278 | 111.2 | 103 |
| 500 | 1061 | 403 | 80.6 | 91.6 |
| 1000 | 1579 | 921 | 92.1 | 95.2 |

TABLE 2

| | AVG | | |
|---|---|---|---|
| | (OD 490 nm) | SD | CV(%) |
| Apoe | 0.39 | 0.026 | 6.7 |
| Ldlr1 | 0.25 | 0.011 | 4.4 |
| Ldlr2 | 0.31 | 0.015 | 4.8 |
| Ldlr3 | 0.25 | 0.016 | 6.4 |
| Ldlr4 | 0.18 | 0.012 | 6.7 |
| Ldlr5 | 0.15 | 0.015 | 10 |

### Example 2

### Evaluation of Potential of SAP/oxLDL/β₂-GPI Complex as Atherosclerosis Marker

### Materials and Methods

### (1) Hemodynamics in Mice

200 mL of the SAP/oxLDL/β₂-GPI complex of a known amount (0.8 μg/mL as a concentration equivalent to LDL protein) was administered to the vein of C57BL/6 mice, blood was collected from the ocular fundus over time and centrifuged to recover plasma, and the SAP/oxLDL/β₂-GPI complex was measured by ELISA.

### (2) Changes Over Time

A test was performed using three 10-week-old male LDLR−/− mice. Blood was collected beforehand, then these model mice were given a high fat diet. Then, blood was collected at 12, 14, and 16 weeks old, and the SAP/oxLDL/β₂-GPI complex was measured by ELISA.

Furthermore, a test was separately performed using six 5-week-old male LDLR−/− mice. Blood was collected beforehand, then these model mice were given a high fat diet. Then, blood was collected at 10, 15, and 20 weeks old, and the SAP/oxLDL/β₂-GPI complex was measured by ELISA.

Other materials and methods are the same as in Example 1.

### Results

(1) Hemodynamics of SAP/oxLDL/β₂-GPI complex in mice

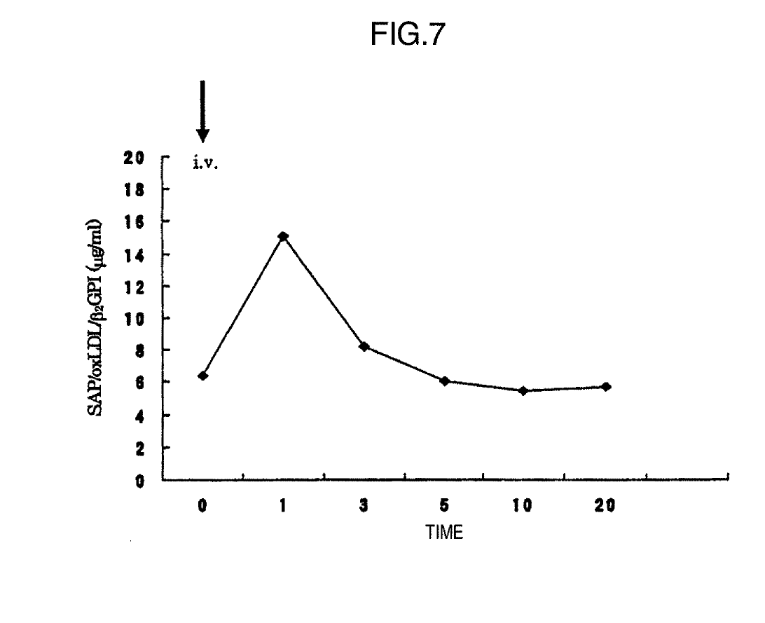

The results are shown in FIGS. 6 and 7. FIGS. 6 and 7 are the results of the separate tests independently performed.

As shown in FIGS. 6 and 7, the SAP/oxLDL/β₂-GPI complex was eliminated from blood within several minutes. Therefore, it was suggested that the SAP/oxLDL/β₂-GPI complex was continuously supplied from the inflammation sites (atherosclerosis lesions) to blood in hyperlipemia model mice given a high fat diet (the complex is detected from blood) (however, the absolute blood concentration varies depending on the administration procedure).

### (2) Changes in SAP/oxLDL/β₂-GPI Complex in LDLR−/− Mice Over Time

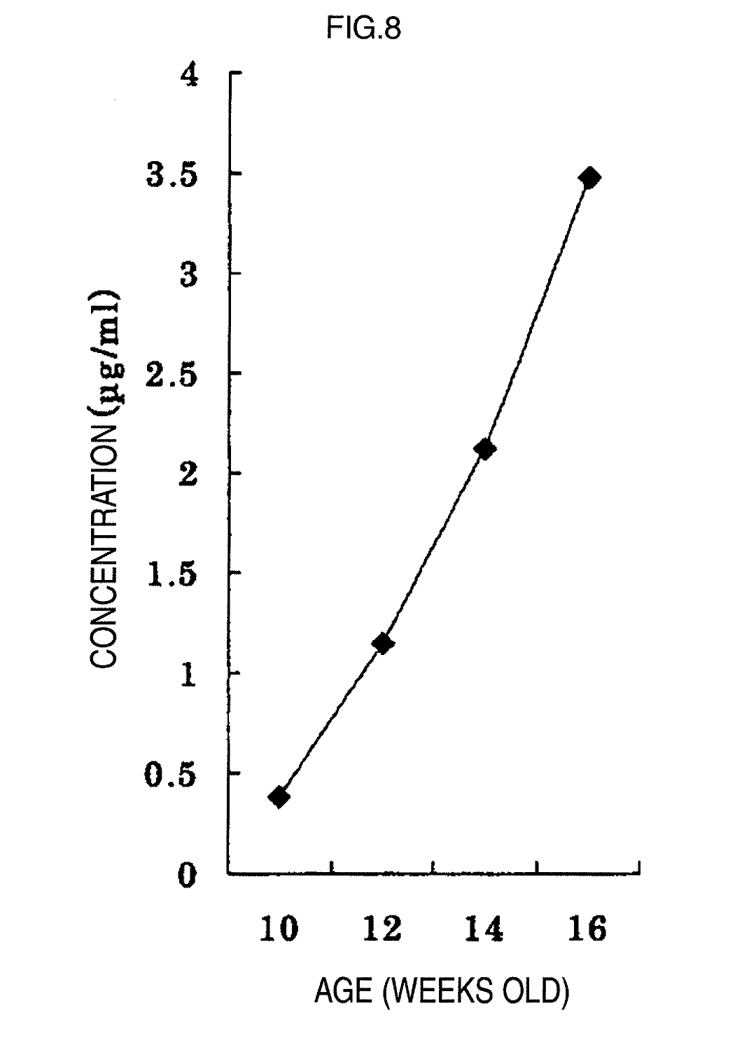

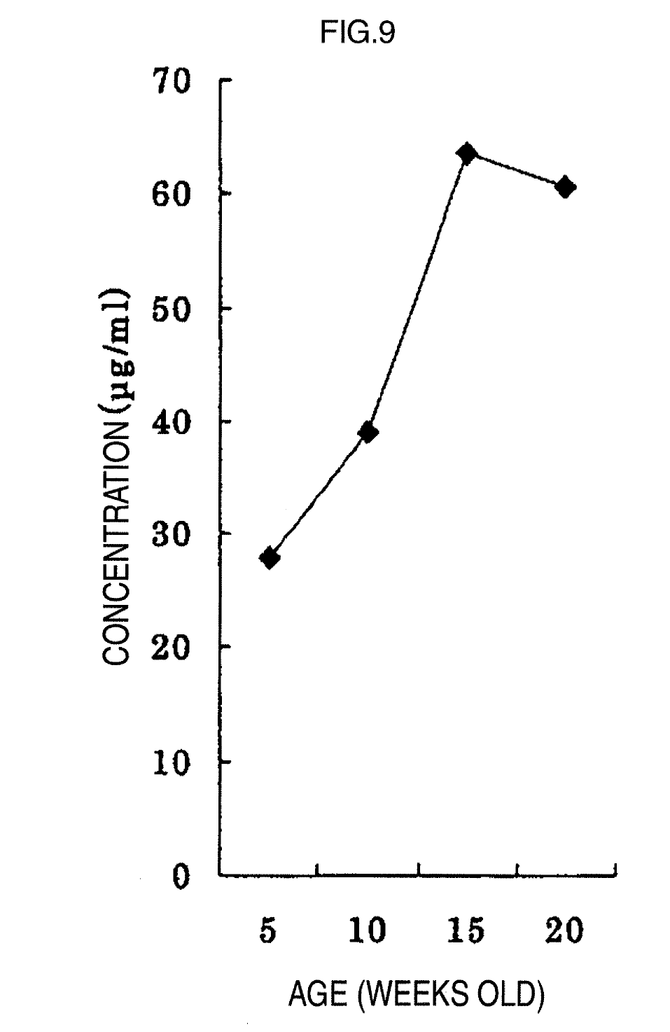

The results (mean values) using 10-week-old mice are shown in FIG. 8, and the results (mean values) using 5-week-old mice are shown in FIG. 9. As shown in FIGS. 8 and 9, the amount of the SAP/oxLDL/β₂-GPI complex in blood increased over time. This result suggested that increases of the SAP/oxLDL/β₂-GPI complex was associated with plaque formation with aging.

### Example 3

### Detection of Mouse Sap/oxLDL Complex and Drawing of Standard Curve

The SAP/oxLDL/β₂-GPI complex prepared in Example 1 was serially diluted and measured by ELISA described in Example 1 (however, using an antibody solid-phased by adding a mouse monoclonal anti-oxLDL antibody [10 μg/mL solution] instead of WB-CAL-1 onto a solid phase at 50 μL/well). A standard curve was drawn for the relationship between the concentrations of the SAP/oxLDL complex measured (“part in the SAP/oxLDL/β₂-GPI complex in which SAP and oxLDL are forming a complex”) and the measurement results.

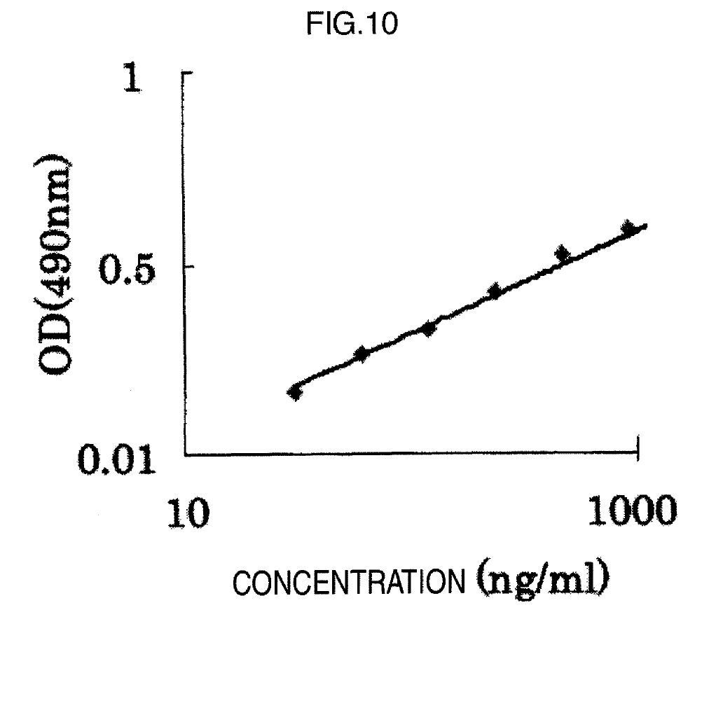

The results are shown in FIG. 10. As shown in FIG. 10, favorable linearity was confirmed between the concentrations and the measured values of the SAP/oxLDL complex, and a standard curve could be drawn. Therefore, it was demonstrated that the SAP/oxLDL complex could be specifically detected in this assay system.

### Example 4

### Detection of Human SAP/oxLDL/β₂-GPI Complex, Drawing of Standard Curve, and Measurement of Complex in Patient Serum

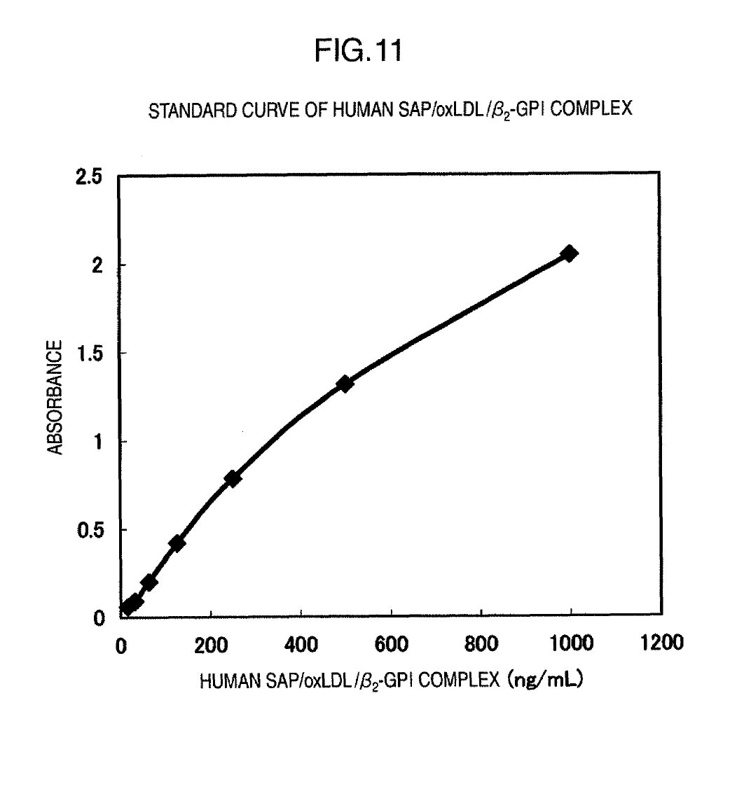

As with the above-mentioned mouse SAP/oxLDL/β₂-GPI complex, the human SAP/oxLDL/β₂-GPI complex was measured by ELISA. FIG. 11 shows the obtained standard curve. Furthermore, the complex in 250-fold diluted serum of normal subjects and patients with acute coronary syndrome (as patients with an arteriosclerotic disease) was measured. The results are shown in Table 3.

As shown in FIG. 11, favorable linearity was confirmed between the concentrations and the measured values of the human SAP/oxLDL/β₂-GPI complex, and a standard curve could be drawn. Therefore, it was demonstrated that the human SAP/oxLDL/β₂-GPI complex could be specifically detected in this assay system.

Furthermore, as shown in the results in Table 3, although only a few samples are shown, the concentrations of the human SAP/oxLDL/β₂-GPI complex in serum were higher in the disease group than in normal subjects. Therefore, it was found that atherosclerosis or the like could be detected by measuring the human SAP/oxLDL/β₂-GPI complex.

TABLE 3

| | Complex concentration |
|---|---|
| Serum | (absorbance) |
| Healthy subjects 1 | 0.194 |
| Healthy subjects 2 | 0.103 |
| Patients with acute coronary syndrome 1 | 0.489 |
| Patients with acute coronary syndrome 2 | 0.305 |

### Example 5

### Preparation of Kit of the Present Invention

(1) The kit of the present invention (ELISA kit) comprising the following was prepared.
1. 96-well immunoplate 1
2. Anti-β₂-GPI antibody (WB-CAL-1: for solid-phasing onto immunoplate) 1
3. Sheep anti-SAP antibody (primary antibody) 1
4. HRP-labeled anti-sheep IgG antibody (secondary antibody) 1
5. o-PD reagent solution 1
6. SAP/oxLDL/β₂-GPI complex (standard)
(2) The kit of the present invention (ELISA kit) comprising the following was prepared.
1. 96-well immunoplate on which anti-β₂-GPI antibody (WB-CAL-1) is immobilized 1
2. Anti-SAP antibody (primary antibody) 1
3. HRP-labeled anti-immunoglobulin antibody (secondary antibody) 1
4. o-PD reagent solution 1
5. SAP/oxLDL/β₂-GPI complex (standard)
(3) The kit of the present invention (ELISA kit) comprising the following was prepared.
1. 96-well immunoplate on which anti-β₂-GPI antibody (WB-CAL-1) is immobilized 1
2. HRP-labeled anti-SAP antibody 1
3. DAB solution 1
4. SAP/oxLDL/β₂-GPI complex (standard)
(4) The kit of the present invention 2 (ELISA kit) comprising the following was prepared.
1. 96-well immunoplate 1
2. Anti-oxLDL antibody (for solid-phasing onto immunoplate) 1
3. Sheep anti-SAP antibody (primary antibody) 1
4. HRP-labeled anti-sheep IgG antibody (secondary antibody) 1
5. o-PD reagent solution 1
6. SAP/oxLDL complex (standard)
(5) The kit of the present invention 2 (ELISA kit) comprising the following was prepared.
1. 96-well immunoplate on which anti-oxLDL antibody is immobilized 1
2. Anti-SAP antibody (primary antibody) 1
3. HRP-labeled anti-immunoglobulin antibody (secondary antibody) 1
4. o-PD reagent solution 1
5. SAP/oxLDL complex (standard)
(6) The kit of the present invention 2 (ELISA kit) comprising the following was prepared.
1. 96-well immunoplate on which anti-oxLDL antibody is immobilized 1
2. HRP-labeled anti-SAP antibody 1
3. DAB solution 1
4. SAP/oxLDL complex (standard)

### BRIEF DESCRIPTION OF THE DRAWINGS

FIG. 1 is a graph showing the standard curve of the complex of the present invention;

FIG. 2 is a graph showing changes in formation of the complex of the present invention over time (detected by ELISA);

FIG. 3 is a photo showing changes in formation of the complex of the present invention over time (detected by electrophoresis);

FIG. 4 is a graph showing the detection results of the complex of the present invention in blood of the hyperlipemia model mice given a high fat diet;

FIG. 5 is a graph showing results of the reliability test (dilution linearity test) of ELISA used for detection of the complex of the present invention;

FIG. 6 is a graph showing hemodynamics of the complex of the present invention;

FIG. 7 is a graph showing hemodynamics of the complex of the present invention;

FIG. 8 is a graph showing changes in the complex of the present invention in blood over time;

FIG. 9 is a graph showing changes in the complex of the present invention in blood over time;

FIG. 10 is a graph showing the standard curve of the complex of the present invention 2 (“part where SAP and oxLDL form a complex” in the SAP/oxLDL/β₂-GPI complex); and

FIG. 11 is a graph showing the standard curve of the complex of the present invention (human SAP/oxLDL/β₂-GPI complex).

1. A complex comprising serum amyloid P component, oxidized LDL, and β₂-glycoprotein I.

2. A method for detecting the complex according to claim 1, comprising a step of bringing at least one of an anti-serum amyloid P component antibody and an anti-β₂-glycoprotein I antibody into contact with a sample that may contain the complex according to claim 1.

3. The method for detecting the complex according to claim 2, comprising a step of bringing an anti-serum amyloid P component antibody and an anti-β₂-glycoprotein I antibody into contact with a sample that may contain the complex according to claim 1 to form a sandwich complex comprising “anti-serum amyloid P component antibody—the complex according to claim 1—anti-β₂-glycoprotein I antibody”.

4. The detection method according to claim 2, wherein at least one of the anti-serum amyloid P component antibody and the anti-β₂-glycoprotein I antibody is immobilized on a solid phase.

5. The detection method according to claim 4, further comprising the following steps (1) to (3):
step (1), a step of bringing a sample that may contain the complex according to claim 1 into contact with a solid phase on which antibody A is immobilized to form a first complex represented by “antibody A immobilized on a solid phase—the complex according to claim 1”;
step (2), a step of bringing antibody B into contact with the first complex formed in step 1 to form a sandwich complex represented by “antibody A immobilized on a solid phase—the complex according to claim 1—antibody B”; and
step (3), a step of detecting the sandwich complex formed in step 2,
wherein, the antibody A refers to one of an anti-serum amyloid P component antibody and an anti-β₂-glycoprotein I antibody, and the antibody B refers to the other antibody.

6. The detection method according to claim 2, wherein the sample that may contain the complex according to claim 1 is a sample derived from an organism.

7. The detection method according to claim 6, wherein the sample derived from an organism is a body fluid.

8. A disease detection method, comprising:
step (1), a step of detecting the complex according to claim 1 present in a body fluid by the method according to claim 6 or 7; and
step (2), a step of associating a detection result in step (1) and a disease.

9. The detection method according to claim 8, wherein the disease is selected from hyperlipemia and atherosclerosis.

10. A kit for detecting the complex according to claim 1, comprising an anti-serum amyloid P component antibody and an anti-β₂-glycoprotein I antibody.

11. The detecting kit according to claim 10, wherein at least one of the anti-serum amyloid P component antibody or the anti-β₂-glycoprotein I antibody is immobilized on a solid phase.

12. (canceled)

13. The detecting kit according to claim 10, wherein the disease is selected from hyperlipemia and atherosclerosis.

14. A complex comprising serum amyloid P component and oxidized LDL.

15. A method for detecting the complex according to claim 14, comprising a step of bringing at least one of an anti-serum amyloid P component antibody and an anti-oxidized LDL antibody into contact with a sample that may contain the complex according to claim 14.

16. The method for detecting the complex according to claim 15, comprising a step of bringing an anti-serum amyloid P component antibody and an anti-oxidized LDL antibody into contact with a sample that may contain the complex according to claim 14 to form a sandwich complex comprising “anti-serum amyloid P component antibody—the complex according to claim 14—anti-oxidized LDL antibody”.

17. The detection method according to claim 15, wherein at least one of the anti-serum amyloid P component antibody and the anti-oxidized LDL antibody is immobilized on a solid phase.

18. The detection method according to claim 17, comprising:
step (1), a step of bringing a sample that may contain the complex according to claim 14 into contact with a solid phase on which antibody C is immobilized to form a first complex represented by “antibody C immobilized on a solid phase—the complex according to claim 14”;
step (2), a step of bringing antibody D into contact with the first complex formed in step 1 to form a sandwich complex represented by “antibody C immobilized on a solid phase—the complex according to claim 14—antibody D”; and
step (3), a step of detecting the sandwich complex formed in step 2,
wherein, the antibody C refers to one of an anti-serum amyloid P component antibody and an anti-oxidized LDL antibody, and the antibody D refers to the other antibody.

19.-26. (canceled)
